(19) United States
(12) Patent Application Publication
(10) Pub. No.: US 2006/0269592 A1
(43) Pub. Date: Nov. 30, 2006

(54) Discrete patch for viral lesions

(76) Inventor: William Hart, Freehold, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/439,860

(22) Filed: May 24, 2006

Related U.S. Application Data
(60) Provisional application No. 60/685,603, filed on May 27, 2005.

(51) Int. Cl. A61F 13/02; A61L 15/16

(52) U.S. Cl. 424/448

(57) ABSTRACT

The invention is a discrete patch suitable for use in the treatment of a viral lesion, which patch includes a backing layer and an adhesive layer, where the adhesive layer is substantially free of hydrocolloid particles, and the patch has a thickness ranging from about 10 microns to about 1,500 microns and is substantially free of topical anti-acne agents, and methods for treating viral lesions where a discrete patch of the invention is applied to a viral lesion and maintained in contact therewith for a time effective to substantially complete re-epithelialization of the lesion.

### FIELD OF THE INVENTION

The present invention relates to a discrete patch for viral lesions, such as cold sores, and to a method for treating viral lesions.

### BACKGROUND OF THE INVENTION

Herpes simplex viruses are known to cause open lesions or sores on those afflicted with the disease. Herpes simplex-1 virus is known to cause cold sores around the mouth. Cold sores are characterized by an initial itching or burning phase, followed by a visible outbreak of the viral lesion. The lesion is moist and if not covered will dry to a crust that is itchy and can crack. The duration of outbreak usually lasts 1 to 2 weeks. Herpes simplex-2 is known to cause genital sores. Other viral outbreaks., such as shingles or chicken pox, are caused by herpes zoster. The lesions or sores (hereafter “lesions”) associated with Herpes viruses tend to dry out and crack over time and can be quite painful. Those afflicted with the virus often tend to be embarrassed by the appearance of the lesions. Therefore, there is a need for a discrete covering for viral lesions that protects the lesion and promotes healing of the lesion.

U.S. Pat. No. 6,495,158 teaches an acne patch. The patch includes a backing material, a hydrophobic sizing agent covering at least a portion of the backing material, an adhesive for adhering the patch to the skin, and an anti-acne agent. The adhesive may include hydrophilic polymers.

Despite the teaching of the prior art, there is a continuing need for a discrete covering for viral lesions that protects the lesion and promotes healing of the lesion.

### SUMMARY OF THE INVENTION

The present invention relates to an adhesive patch for covering a portion of the anatomical surface of a human being, for example a viral lesion, said patch being able to adhere to the skin or mucosa, and/or a wound, said patch comprising a backing layer and a layer of a skin-friendly adhesive that is substantially free of hydrocolloid particles for adhering the patch to the skin or mucosa. The patch has a thickness of from about 30 microns to about 1,500 microns and may be free of, or substantially free of, topical anti-acne agents. By substantially free of topical anti-acne agents, it is meant that the patch does not include such agents in amounts effective to prevent or treat acne, for example less than about 0.3 percent by weight. The patch may consist essentially of the backing layer and the adhesive layer. In a second embodiment, the present invention provides a method for treating a viral lesion including covering a viral lesion with a patch according to the present invention and maintaining the patch in contact with the lesion for a period of time effective to substantially complete re-epithelialization of the lesion.

### DETAILED DESCRIPTION OF THE INVENTION

The present invention provides a non-occlusive adhesive patch, i.e. one that will enable moisture on the surface of the skin to evaporate through the patch so as to prevent the undesired accumulation of moisture, which, if occurs, could cause the patch to detach from the skin or even facilitate the growth of bacteria beneath the patch. As used herein, the term patch means an adhesive coated backing material that does not include an absorbent structure or layer, such as a wound-contacting pad used in conventional wound dressings. The invention provides a lighter, more flexible and less obtrusive patch, while still providing the excellent wear time and healing properties required for healing of moist wounds, such as viral lesions in areas such as the lip region. The patch may consist essentially of the backing sheet, or layer, and an adhesive layer, and excludes any third element that attributes absorbency to the patch. For example, the patch is substantially free of hydrocolloid particle or absorbent fibers contained in the adhesive layer, absorbent pads, and the like.

The patches of the present invention are intended to moderate the formation of blisters and prevent the formation of a crust. The absence of crust seems to reduce the healing time and cause less inconvenience for the patient. Secondary infections are avoided, which also contributes to reduce healing time. Consumers typically wear the patch for from about 12 hours to about 24 hours and then replace it. The patch of the present invention protects the lesion from being touched and from secondary infection. The patch is discrete, not being easily noticed by other people. By virtue of its occlusive nature the product provides soothing relief from itching, burning, and pain associated with the lesion.

Due to the high permeability of the patch of the present invention the patch may not need to be able to store large amounts of exudates/moisture. However, due to evaporation, the overall moisture handling capacity will be high, hence the flux of moisture through the patch is high. The thickness of the dressing is often selected based on the amount of exudates expected from the wound, i.e. a thick dressing for a highly exuding wound. The thickness of the adhesive may be in the range of 20-300 microns. The vapor permeability of the backing sheet may be in the range of 200-6,000 g/m²/day. The thickness of the backing sheet may be in the range of 10-1,000 microns. It has been found that a backing sheet with such thickness and vapor permeability provides a non-occlusive patch. Moreover, the overall low thickness of the patch, results in a discrete appearance once applied to the application site.

The patch of the present invention may have a wear time on skin of at least 8 hours, more preferred at least 12 hours, and even more preferred at least 24 hours. When applied to the mucosa and other places where the patch is exposed to high humidity and friction, such as in the lip region, the wear time may naturally be shorter. For application to the lip region, the wear time is preferably at least 2 hours, more preferred at least 3 hours and most preferred at least 4 hours. The patch is applied to the lesion and contact with a patch then is maintained for a time effective to substantially complete re-epithelialization of the lesion.

The patch is coated with an adhesive on one side. The adhesive coated side is used to adhere the patch or cover to the skin of the consumer. The adhesives are substantially free of hydrocolloid particles. As used herein, substantially free of hydrocolloid particles means that the adhesive contains less than about 1 percent by weight of hydrocolloid particles based on the total weight of the adhesive composition; for example, less than about 0.5 percent, or less than about 0.1 percent by weight. Suitable adhesives include, but are not limited to, those based on styrenic block copolymers and tackifying resins such as HL-1491 available from HB-Fuller Co. (St. Paul, Minn.), H-2543 available from ATO-Findley (Wawatausa, Wis.), and Resyn 34-5534 available from National Starch & Chemical Company (Bridgewater, N.J.). Ethylene copolymers, including ethylene vinyl acetate copolymers, are also useful as adhesives. Silicone adhesives, such as those provided by DOW Chemical Corporation are also useful.

The adhesive of the patch of the invention may be any suitable skin-friendly adhesive. The skin-friendly adhesive may be any skin-friendly adhesive known per se for production of medical articles that are to be adhered to human skin. Suitable adhesives include solvent-based, acrylic-based, dextrin-based, and urethane-based adhesives, as well as natural and synthetic elastomers. The adhesives may also include amorphous polyolefins, including amorphous polypropylene, such as HL-1308 available from HB Fuller or Rextac RT 2373 available from Huntsman (Odesssa, Tex.). The adhesive may be based on Kraton® Brand synthetic elastomers, or natural rubber. These adhesives may also include tackifiers, anti-oxidants, processing oils, and the like as is known in the art. The adhesives may be aqueous or solvent-based adhesives, or they may be hot melt adhesives, as desired. The adhesive can be applied in any desired manner, e.g., by spraying, screen printing or slot die coating. The amount of adhesive typically applied may vary from a basis weight of about 20 grams per square meter (“gsm”) to about 100 gsm. Basis weight, as used herein, is related to thickness and the terms gsm and microns may be used interchangeably. One gsm is approximately equal to 30 microns.

Though the term adhesive is used herein, it is understood that the term may cover any substance having adherent properties, such as adhesives, silicone or rubbery substances, petrolatum or the like. The adhesive may be a pressure sensitive adhesive of any suitable kind known per se. The thickness of the adhesive layer of the patch of the present invention may be substantially constant over the surface, or the patch may have a thicker portion at the center of the patch, surrounded by a thinner periphery, i.e. a beveled edge. It has surprisingly been shown that a better performance for the patch is achieved by having a thin edge portion. The thin periphery or the patch decreases the risk of rolling-up of the edge portion. The rolling up of the edge portion may lead to reduced wear time and is undesired. Furthermore, the thin edge portion is less exposed to water coming from outside, and renders it possible to obtain an extreme water-block. The thickness of the adhesive layer may be 20-200 microns, or 25-150 microns, or 30-100 microns, or even 50-80 microns.

It may be preferred that the patch has a substantially uniform thickness. Due to the low thickness, beveling may not be necessary in order to ensure good tack and reduce rolling up of the edge portions. In one embodiment of the invention the patch is 100-200 microns thick. The obtained patch is thus thick enough to be handled without folding or wrinkling but at the same time remarkably thinner than traditional hydrocolloid dressings. Due to the permeability, this patch may be suitable for use on scratches and wounds, which normally would be treated with a thicker patch. It may be suitable for such minor wounds or skin damages, where the high moisture capacity of a traditional thick hydrocolloid is not necessary. The patch of the present invention easily follows the movements of the skin and, furthermore, it hardly takes up any place, which may be important when worn on the foot or toes being placed in a snug shoe. A thin layer of adhesive is desired, as this will reduce the overall thickness of the patch.

The thinner the dressing or patch is, the more flexible and more capable of following the movements of the body it is and the more discrete it appears. The dressing or patch is especially suitable for use in the face or other visible or exposed areas of the body and it may therefore be desired that the patch blends into the skin and appears almost invisible

The surface area of the backing sheet may e.g. be 5-25 cm², such as 10-20 cm², or smaller, such as less than 5 cm², such as at most 4 cm², such as at most 2 cm², such as in the range of 1-2 cm², or smaller, such as 0.08-1 cm², such as 0.1-0.8 cm², such as 0.12-5 cm². For facial application, e.g. of a thin film patch, the surface area is usually less than 5 cm².

In one embodiment of the invention the patch may have beveled edges. The beveling may provide a smoother transition between the patch and the skin, rendering the patch more invisible. The outer periphery of the patch is preferably beveled in analogy with the disclosure of U.S. Pat. No. 4,867,748 or U.S. Pat. No. 5,133,821, the contents each of which are incorporated herein by reference, in order to reduce the risk of “rolling-up” the edge of the patch reducing the wear-time. The edge is preferably beveled so that the thickness adjacent to the edge does not exceed about 30% of the maximum thickness of the patch, or not exceeding 25% of the maximum thickness.

The patch of the invention has surprisingly good water resistance, and beveling of the edge may further enhance these properties. By the expression “water resistance” is understood that after application of the patch to the body part, the patch is capable of resisting water or humidity, such as bathing hand washing, swimming or perspiration. The excellent water resistance and the good moisture handling qualities render it possible to achieve an extremely long wear time for the patch compared to commonly known products. By beveling the edges of the patch to a very low thickness, or having an overall low thickness of the patch, the wear time may be increased. The rolling-up of the edges of the patch during use may depend on the amount of exposed adhesive along the edge portion, so, the thinner layer of adhesive along the edge, the less rolling-up may occur.

The patch may be prepared by a one step process, where a high flexibility in production may be achieved, thin bevelings may be prepared, with adapted center thickness, and at the same time sufficient capacity to handle exudates from a wound or blister.

The adhesive layer may be in the form of a pattern, such as geometric pattern or a random pattern, or the adhesive layer may comprise larger interruptions in the form of areas with no adhesive, e.g. the central part of the patch. It is preferred that the adhesive layer is uninterrupted. The uninterrupted layer provides several advantages, such a less wrinkling, better invisibility and better blending into the skin. The backing layer being uncoated with adhesive may be more non-transparent and thus more visible. Furthermore, the pattern may leave marks on the skin when the patch is, removed. The adhesive patch may be in a flat continuous layer.

The article of the present invention is discrete. As used herein, discrete means that the cover is not visually apparent to other people. Both the size of the backing layer and the color of the backing layer may affect the discrete nature of the patch. Most backing materials known in the wound care art are suitable for use in the present invention. Transparent or translucent backing materials and backing materials having colors that match skin color are preferred. The colors may be inherent in the polymer that the backing material is made from. Alternatively, pigments may be added to the polymer and blended to provide backing materials of colors that match skin colors. Suitable backing materials include, but are not limited to, polyolefin films, such as polyethylene and polypropylene, polyvinylchloride films, polyetheramide films, polyamide films, polyester films, ethylene vinyl acetate films, woven fabrics, nonwoven fabrics, foams and polyurethane films.

Suitable backings or films should be flexible so that when they are applied to skin, they move and flex with the skin so as to be comfortable to the wearer. The backing material may be monolithic (non-apertured), microporous, or apertured. It is suitable that the backing layer is a substantially water-impervious film that protects the adhesive from being adversely affected when the wearer is bathing or in case of incidental wetting of the area. The backing material has a moisture vapor transmission rate (“MVTR”) of from about 200 gms/24 hours to about 6,000 gms/24 hours, or from about 800 gms/24 hours to about 1,300 gms/24 hours. The backing material and adhesive are selected such that the MVTR of the entire patch ranges from 200 gms/24 hours to about 6,000 gms/24 hours, or from about 500 gms/24 hours to about 1,300 gms/24 hours.

In accordance with the invention, it has been found in practice that when a thinner backing layer or film is used than is normally used when preparing medical dressings, an improved stretchability and adaptability is obtained at the same time as the modulus is reduced. The backing layer may preferably be an elastic, flexible and non-sticking film that protects the adhesive during storage as well as during use. The water impervious, but vapor permeable layer or film is preferably a low-friction flexible polymer film reducing the risk of unwanted stress in the area of application. The backing layer may have a suitable thickness for the intended use. If the patch were desired for an “invisible” face patch, a rather thin film would be appropriate. It is preferred that the backing layer has a thickness of less than 25 microns. A preferred thickness of this film may be below 20 microns, more preferred about 10-18 microns, e.g. about 15 microns, thus resulting in a significant decrease of the modulus, compared to a film that is normally used when preparing medical dressings. An improved stretchability and adaptability is obtained at the same time as the modulus is reduced.

In accordance with a preferred embodiment the backing layer is a film showing a low surface friction. Furthermore, the surface may be opaque, with a reflection being near to the reflection of skin, thus enabling the patch to blend with the skin color and reflection and be less visible. The backing layer may be colored in suitable colors, e.g. flesh-color. The backing layer may be transparent, translucent, opaque or non-transparent, depending on the intended use.

The patch of the invention is optionally covered in part or fully by one or more release liners or cover films to be removed before or during application. A protective cover or release liner may for instance be siliconized paper. It does not need to have the same contour as the patch, e.g. a number of patches may be attached to a larger sheet of protective cover. The protective cover is not present during the use of the patch of the invention and is therefore not an essential part of the invention. Furthermore, the patch of the invention may comprise one or more “non touch” grip(s) known per se for applying the patch to the skin without touching the adhesive layer. Such a non-touch grip is not present after application of the patch. For larger patches it is suitable to have 2 or 3 or even 4 “non-touch” grips.

The patch of the invention may further comprise one or more cover layers. The cover layer may protect the patch during storage and help easy application of the patch. The cover layer is removed during or after application. Preferably, in all embodiments of the present invention, the patch is provided in the form of a backing layer with an adhesive applied to one surface thereof. The adhering surface of the patch may comprise a pharmaceutically active substance. For example, emollients or e.g. retinoids for treating or preventing formation of psoriasis, eczema, callous, skin, corns or blisters. Examples of applicable pharmaceutical medicaments include a cytochine, such as a growth hormone or a polypetide growth factor such as TGF, FGF, PDGF, EGF, IGF-1, IGF-2, colony stimulating factor, transforming growth factor, nerve stimulating growth factor and the like giving rise to the incorporation of such active substances in a form being apt to local application in a wound in which the medicament may exercise its effect on the wound, other medicaments such as bacteriostatic or bactericidal compounds, e.g. iodine, iodopovidone complexes, chloramine, chlorhexidine, silver salts such as sulphadizine, silver nitrate, silver acetate, silver lactate, silver sulphate, silver sodium thiosulphate or silver chloride, zind or salts thereof metronidazol, sulpha drugs, and pencillins, tissue-healing enhancing agents, e.g. RGD tripeptides and the like, proteins, amino acids such as taurine, vitamins such as ascorbic acid, enzymes for cleansing of wounds, e.g. pepsin, trypsin and the like, proteinase inhibitors for use in e.g. surgical insertion of the dressing in cancer tissue and/or other therapeutic agents which optionally be used for topical application, pain relieving agents such as NSAIDS, lidocaine or chinchocaine, emollients, retinoids or agents having a cooling effect.

The cover of the present invention is discrete. Therefore, the size and shape of the cover is designed to minimize the visibility of the cover to others. The thickness of the cover may range from about 0.050 mm to about 0.30 mm, and is preferably from about 0.065 mm to about 0.10 mm. In one embodiment, the thickness of the cover is 0.085 mm. The size and shape of the cover may vary depending on the particular use and location of the lesion to be covered. The shape and size of the cover must be such that it effectively covers and provides continued adhesion to the lesion, while remaining discreet. For example, when the cover is applied to a viral lesion in the area of the mouth, such as on or in close proximity to the lip, it is advantageous that the shape of the cover is a circle. The diameter of the circular cover may range from about 5 mm to about 25 mm, preferably from about 10 mm to about 20 mm. In one embodiment, the diameter of the circular cover is 15 mm.

While additional medicaments for treatment of viral lesions are not required in certain embodiments of the present invention, in other embodiments the patches may optionally include medicaments known per se for such purposes being contained in the adhesive or being applied thereto. Suitable anti-viral medicaments for the treatment of herpes may, for example, comprise aciclovir or penciclovir. Azelain acid or isotretinoin may be used in a medicament for the treatment of acne. In respect of the treatment of warts, a mitotic inhibitor, such as podophyllotoxin, is applicable. Warts and/or callous skin may be treated by salicylic acid-based medicaments. Patches for treatment of acne, scratches or wounds may e.g. comprise antiseptic/antibiotic substances, vitamin compounds or other wound healing substances. The above mentioned pharmaceutically active substances may be applied to the adhering surface of the dressing sheet after completion of the adhering coating, or they may be mixed into the adhesive prior to coating thereof onto the backing layer. In one embodiment of the invention the medicament may be applied to the patch before application. An amount of a gel or cream may be applied to the central part of the patch before application to the treatment site. In the treatment of Herpes it may be advantageous to apply an Acyclovir containing cream or gel such a Zovir before application to the Herpes site.

### EXAMPLES

Several Examples are set forth below. The claims should not be considered to be limited to the details thereof.

### Example 1

Lintec Ohkura 85NES50 clear polyurethane film (50 micron thick) was coated with LS486H solvent based acrylic adhesive that was free of hydrocolloid particles at a coat weight of 30 g/m². A Tekkote silicone release liner was applied to the coated film. The laminate was cut into circular shaped bandages of the present invention having a diameter of 20 mm.

### Example 2

Clear perforated tri-layer ABA polyethylene outer layers and amorphous polyolefin inner layer film (code 27936, target thickness of 100 microns) was coated with Bostik Findley HM321-02 hot melt adhesive that was free of hydrocolloid particles at a coat weight of 50 g/m². A Tekkote silicone release liner was applied to the coated film. The laminate was cut into circular shaped bandages of the present invention having a diameter of 20 mm.

### Example 3

Smith & Nephew Opaque biaxial embossed PBA 74 polyurethane blended with high impact polystyrene film (basis weight 105 g/m²) was coated with A16 solvent based Acrylic adhesive that was substantially free of hydrocolloid particles (ID31/EU31B National Duro Tak adhesive) at a minimal coat weight of 30 g/m². A Tekkote silicone release liner was applied to the coated film. The laminate was cut into circular shaped bandages of the present invention having a diameter of 15 mm.

This was a single-center, evaluator-blinded, randomized, parallel-group study. Subjects with a history of recurrent Herpes labialis (RHL) were assigned to one of three treatment groups, to use one of three investigational cold sore patches. Subjects were instructed to return to the study center within 24 hours of first symptoms for clinical evaluations. A designated investigator, who was blinded to treatment assignments of all subjects, conducted clinical assessments at each study visit. Subjects completed a daily diary to report symptom evaluations and patch usage and returned to the center when the lesion was completely healed.

The primary endpoint was patient-assessed and clinician confirmed time to complete healing defined as time to achieve substantially complete re-epithelialization of lesion with patch treatment compared to no treatment. By substantially complete re-epithelialization, it is meant that the lesion has been reduced to the extent where it is not clearly noticeable and the subject is no longer experiencing significant symptoms of the lesion.

The secondary endpoint was quality of healing consisting of the following, for all treatments. Subjects were provided surveys in which they were asked to rate the following with respect to prior experiences without the patches of the present invention: redness, itching, burning, tenderness, discomfort, pain, swelling, and formation of crust around the sores.

At the randomization visit, subjects were asked to lightly blot their lips with a tissue before each visual evaluation. The visual evaluator confirmed subject eligibility (active presence of cold sore on lips) and assigned a grade or stage to the test site. Subjects were randomized to evaluate a single test article only. Each subject was instructed on the appropriate method of product application and subjects performed the initial application under the supervision of the instructor.

Subjects were instructed by the study staff on the required frequency of application, use conditions and study restrictions. All subjects were provided a supply of test article sufficient to last for the duration of the study, a diary to record each product's application time which included a self-evaluation for possible tingling, itching, burning, pain, tenderness or discomfort. Subjects were instructed to perform all the subsequent applications at home.

On the morning of the end of treatment/healed lesion visit, no patch application was made prior to the final evaluations at the test center. The final evaluations were completed by the center staff to determine the healing of the cold sore.

At the final visit, diaries were collected and reviewed by the investigative staff and remaining test product was collected. Adverse events were assessed and concomitant medications were reviewed.

Results

The patches of the present invention tended to stay in place well. On average, the patches tended to stay on for about 12 hours. There were some occasions when the patches required replacement due to showering, washing the face, or brushing the teeth. Overall, although some symptomatic indicators were worse on some days for some subjects, the patches were rated favorably with respect to redness, itching, burning, tenderness, discomfort, pain, and swelling of the sore and the development of crust.

We claim:

1. A discrete patch for a viral lesion, comprising:
a backing layer; and
an adhesive layer, wherein said adhesive layer is substantially free of hydrocolloid particles, wherein said patch has a thickness ranging from about 10 microns to about 1,500 microns and is substantially free of topical anti-acne agents.

2. The patch according to claim 1 wherein the backing material is selected from the group consisting of polyolefin films, polyvinylchloride films, polyetheramide films, polyamide films, polyester films, ethylene vinyl acetate films, woven fabrics, nonwoven fabrics, foams, and polyurethane films.

3. The patch according to claim 1 wherein the adhesive is selected from the group consisting of styrenic block copolymers and tackifying resins, ethylene copolymers, ethylene vinyl acetate copolymers, silicone adhesives, solvent based acrylic polymers, dextrin based adhesives, urethane based adhesives, and amorphous polyolefins.

4. The patch according to claim 1 wherein the patch has a moisture vapor transmission rate of from 200 g/m² day to 6,000 g/m² day.

5. The patch according to claim 1 wherein the patch has a moisture vapor transmission rate of from 500 g/m²day to 1,300 g/m²/day.

6. The patch according to claim 1 wherein said patch consists essentially of said backing layer and said adhesive layer.

7. A method for treating a viral lesion comprising:
applying a patch to a viral lesion, said patch comprising a backing layer and an adhesive layer, wherein the adhesive layer is substantially free of hydrocolloid particles, the patch has a thickness ranging from about 10 microns to about 1,500 microns, and said patch is substantially free of topical anti-acne agents; and
maintaining said patch in contact with said viral lesion for a time effective to substantially complete re-epithelialization of said lesion.

8. The method according to claim 7 wherein the backing material is selected from the group consisting of polyolefin films, polyvinylchloride films, polyetheramide films, polyamide films, polyester films, ethylene vinyl acetate films, woven fabrics, nonwoven fabrics, foams, and polyurethane films.

9. The method according to claim 7 wherein the adhesive is selected from the group consisting of styrenic block copolymers and tackifying resins, ethylene copolymers, ethylene vinyl acetate copolymers, silicone adhesives, solvent based acrylic polymers, dextrin based adhesives, urethane based adhesives, and amorphous polyolefins.

10. The method according to claim 7 wherein the patch has a moisture vapor transmission rate of from 200 g/m²/day to 6,000 g/m²/day.

11. The method according to claim 7 wherein the patch has a moisture vapor transmission rate of from 500 g/m²/day to 1,300 g/m²/day.

12. The method of claim 6 wherein said patch consists essentially of said backing layer and said adhesive layer.
